(19) United States
(12) Patent Application Publication
(10) Pub. No.: US 2025/0321231 A1
(43) Pub. Date: Oct. 16, 2025

(54) METHODS RELATING TO TUMOUR-DERIVED EXTRACELLULAR VESICLES

(71) Applicant: INOVIQ INC., Minneapolis, MN (US)

(72) Inventor: Peter FRENCH, Melbourne, Victoria (AU)

(21) Appl. No.: 18/558,826

(22) PCT Filed: May 6, 2022
(86) PCT No.: PCT/AU2022/050428

§ 371 Date: Nov. 3, 2023

(30) Foreign Application Priority Data
May 6, 2021 (AU) 2021901359

(51) Int. Cl. G01N 33/574; C12N 5/09

(52) U.S. Cl. CPC G01N 33/57492; C12N 5/0693

(57) ABSTRACT

The present disclosure relates to methods of detecting and/or isolating tumour-derived extracellular vesicles from a sample, and populations and compositions comprising the same. Detected and/or isolated tumour-derived extracellular vesicles and compositions comprising the same may be useful in applications such as diagnosing cancer.

### FIELD OF THE INVENTION

The present disclosure relates to methods of detecting and/or isolating tumour-derived extracellular vesicles from a sample, and populations and compositions comprising the same. Detected and/or isolated tumour-derived extracellular vesicles and compositions comprising the same may be useful in applications such as diagnosing cancer.

### BACKGROUND OF THE INVENTION

Small extracellular vesicles (sEV), such as exosomes, are nanovesicles (30-150 nm) released by cells. Tumour cells produce small extracellular vesicles called tumour extracellular vesicles (e.g. tumour exosomes or TEX) which are secreted into the tumour microenvironment in subjects with cancer, or cancer cell cultures.

Tumour exosomes (TEX) are of interest as they appear to be involved in various molecular processes such as suppression of anti-tumour immune responses. Isolating and/or detecting TEX in a mixed population of exosomes is challenging because the TEX and non-TEX are of the same size range and may have common exosome-associated extracellular proteins. Accordingly, classical exosome capture and isolation methods such as ultracentrifugation and size exclusion are not particularly useful means of isolating and/or specifically detecting TEX. Furthermore, the molecular profile of TEX may not be representative of the cell from which they are secreted. For example, in a study by Batista et al., several molecules were found to be enriched (e.g. high mannose, polylactosamine, α2,6-linked sialic acid, complex N-linked glycans) while others were depleted (e.g. terminal blood group A and B antigens) on the exosome surface relative to their parent cells (Batista et al., J Proteome Res. 2011; 10(10): 4624-4633). Accordingly, identification of markers for detection and isolation of TEX remains challenging.

Therefore, there is a need for new methods to detect and/or isolate TEX, in particular for research and diagnostic applications.

### SUMMARY OF THE INVENTION

The present inventors have surprisingly identified that N-glycolylneuraminic acid (Neu5Gc) is expressed on the surface of tumour-derived extracellular vesicles such as exosomes and, accordingly, can be used as a marker to detect and/or isolate tumour-derived extracellular vesicles from a sample.

Accordingly, in a first aspect, the present disclosure encompasses a method of capturing tumour-derived extracellular vesicles from a sample, the method comprising:
contacting a sample comprising tumour-derived extracellular vesicles with a binding molecule that binds to N-glycolylneuraminic acid (Neu5Gc) under conditions that enable binding of the binding molecule to the tumour-derived extracellular vesicles in the sample.

In a second aspect, the present disclosure encompasses a method of isolating tumour-derived extracellular vesicles from a sample, the method comprising:
contacting a sample comprising tumour-derived extracellular vesicles with a binding molecule that binds to N-glycolylneuraminic acid (Neu5Gc) under conditions that enable binding of the binding molecule to the tumour-derived extracellular vesicles in the sample; and
isolating the binding molecule from the sample. In an example, the methods comprise isolating the tumour-derived extracellular vesicles from the sample. In an example, the tumour-derived extracellular vesicles are labelled with a Neu5Gc binding molecule before being isolated from the sample. In an example, the tumour-derived extracellular vesicles are isolated by disassociating them from the Neu5Gc binding molecule using a buffer that can disrupt the binding of Neu5Gc to the binding molecule.

In another aspect, the present disclosure encompasses a method of detecting tumour-derived extracellular vesicles in a sample, the method comprising:
contacting a sample comprising tumour-derived extracellular vesicles with a binding molecule that binds to N-glycolylneuraminic acid (Neu5Gc) under conditions that enable binding of the binding molecule to the tumour-derived extracellular vesicles in the sample; and detecting binding of the binding molecule to tumour-derived extracellular vesicles in the sample, wherein binding of the binding molecule to tumour-derived extracellular vesicles indicates the presence of tumour-derived extracellular vesicles in the sample. In an example, detected tumour-derived extracellular vesicles are isolated using an exosome specific binding molecule such as an antibod(ies) (e.g. anti-CD63).

The present disclosure also provides a binding molecule that binds to N-glycolylneuraminic acid (Neu5Gc) when used for detecting and/or isolating tumour-derived extracellular vesicles from a sample.

The present disclosure further provides a method of isolating tumour-derived extracellular vesicles from a sample, the method comprising:
contacting the sample with a layered, multipolymeric molecular net which comprises multiple layers of at least one binding molecule that binds Neu5Gc under conditions that enable binding of the binding molecule to tumour-derived extracellular vesicles in the sample; and
isolating the layered, multipolymeric molecular net from the sample.

The present disclosure further provides a method of isolating tumour-derived extracellular vesicles from a sample, the method comprising:
contacting the sample with a layered, multipolymeric molecular net which comprises multiple layers of at least one binding molecule that binds exosomes under conditions that enable binding of the molecule to tumour-derived extracellular vesicles in a sample; and isolating the layered, multipolymeric molecular net from a sample to enable detection of Neu5Gc on isolated tumour-derived extracellular vesicles,
wherein, Neu5Gc can be detected using a binding molecule described herein such as an antibody directed against Neu5Gc or a Neu5Gc binding protein derived from the pentameric B sub unit of subtilase cytotoxin (SubAB) from Escherichia coli (SEQ ID NO: 1). In an example, Neu5Gc can be detected using a binding protein having an amino acid sequence as set forth in SEQ ID NO: 2.

The findings of the present inventors also provide the basis for isolating a population or composition of extracellular vesicles enriched for tumour-derived extracellular vesicles. Such populations and compositions may be used in various applications such as basic research or to determine the status and/or progression of a cancer in a subject.

Accordingly, in another aspect, the present disclosure provides a composition comprising extracellular vesicles enriched for extracellular vesicles expressing N-glycolylneuraminic acid (Neu5Gc). In another aspect, the present disclosure also provides a population of extracellular vesicles enriched for extracellular vesicles expressing Neu5Gc, wherein the extracellular vesicles are tumour-derived extracellular vesicles. In an example, the tumour-derived extracellular vesicles are exosomes. In an example, the composition or population is obtained by isolating tumour-derived exosomes via a method disclosed herein.

In an example, at least 20% of the extracellular vesicles in the composition or the population express Neu5Gc. In an example, at least 50% of the extracellular vesicles in the composition or the population express Neu5Gc. In another example, at least 30%, at least 40%, at least 60%, or at least 70% of the extracellular vesicles in the composition or the population are extracellular vesicles expressing Neu5Gc.

In an example, samples used in the methods of the present disclosure may be obtained from a subject suspected of having cancer. In an example, samples used in the methods of the present disclosure may be obtained from a subject that has cancer. In an example, the cancer is selected from a group consisting of breast cancer, melanoma, malignant epithelial tumour, oesophageal carcinoma, gastric cancer, colorectal cancer, epidermoid carcinoma of rectum, pancreatic cancer, hepatocellular carcinoma, lymph node metastases, kidney cancer, urinary bladder cancer, ovarian cancer, uterine cancer, testicular cancer, prostate cancer, neuroblastoma, non-small cell lung cancer, lymphoma, neuroectodermal tumour (astrocytoma and glioblastoma), nephroblastoma (Wilms tumours), sarcoma, Ewing sarcomas and thyroid carcinoma.

In an example, the cancer is breast, ovarian or pancreatic cancer.

In an example, the sample is selected from a group consisting of blood, urine, saliva, faeces, tears, brocho-alveolar lavage fluid (BALF), cerebrospinal fluid (CSF) and seminal fluid. In an example, the sample is a blood sample. In another example, the sample is plasma or serum.

In an example, the sample is a purified or partially purified population of extracellular vesicles. In an example, the purified or partially purified population of extracellular vesicles expresses one or more protein(s) selected from a group consisting of CD63, b2microglobulin, CD11, CD81, CD13, EGFR. In an example, the sample is substantially cell free. In another example, the sample is cell free. In an example, the sample is a tumour cell culture.

In an example, the binding molecule is an isolated protein comprising an amino acid sequence as set forth in SEQ ID NO: 2 or SEQ ID NO: 4 or fragment or variant of SEQ ID NO: 2 or SEQ ID NO: 4 which comprises a modification to at least one of the amino acid sequences in SEQ ID NO: 2 or SEQ ID NO: 4 and, wherein the fragment or variant thereof is capable of binding a2-3-linked N-glycolylneuraminic acid and α2-6-linked N-glycolylneuraminic acid. In an example, the modification comprises a non-conservative substitution or deletion of at least one of the underlined residues of TTSTE. In another example, the modification comprises a deletion of at least one of the underlined residues of TTSTE. In another example, the modification comprises a deletion of both of the underlined residues of TTSTE.

In another example, the binding molecule comprises an amino acid sequence as shown in SEQ ID NO: 2. In another example, the binding molecule comprises an amino acid sequence as shown in SEQ ID NO: 4.

In an example, the binding molecule comprises:
(i) a single chain Fv fragment (scFv);
(ii) a dimeric scFv (di-scFv); or
(iii) a diabody;
(iv) a triabody;
(v) a tetrabody;
(vi) a nanobody
(vii) a Fab;
(viii) a F(ab′)₂;
(ix) a Fv;
(x) an aptamer
(xi) one of (i) to (ix) linked to a constant region of an antibody, Fc or a heavy chain constant domain (C_{H}) 2 and/or C_{H}3;
(xii) one of (i) to (ix) linked to albumin or a functional fragment or variants thereof or a protein that binds to albumin; or
(xiii) an antibody.

In an example, the binding molecule is an antibody.

In another example, the methods of the disclosure further comprise dissociating bound tumour-derived extracellular vesicles from the binding molecule and collecting the dissociated tumour-derived extracellular vesicles. In an example, the method further comprises analysing the tumour-derived extracellular vesicles.

In the above examples, the extracellular vesicles may be selected from a group consisting of small extracellular vesicles, exosomes, exomeres and microvesicles. In an example, the extracellular vesicles are exosomes. In an example, the extracellular vesicles are CD63+ exosomes.

In another example, the present disclosure relates to a method of detecting cancer comprising contacting a sample comprising extracellular vesicles with a binding molecule disclosed herein and, detecting binding of the binding molecule to extracellular vesicles in the sample, wherein binding of the binding molecule to extracellular vesicles indicates the presence of tumour-derived extracellular vesicles in the sample, thereby detecting cancer. In an example, the method of detecting cancer comprises contacting a sample comprising extracellular vesicles with a binding molecule that binds to N-glycolylneuraminic acid (Neu5Gc); and, detecting binding of the Neu5Gc binding molecule to extracellular vesicles in the sample, wherein binding of the Neu5Gc binding molecule to extracellular vesicles indicates the presence of tumour-derived extracellular vesicles in the sample, thereby detecting cancer. In an example, the binding molecule comprises an amino acid sequence as set forth in SEQ ID NO: 2 or SEQ ID NO: 4. However, in other examples, such as those discussed below, the binding molecule may be an antibody.

Any example herein shall be taken to apply mutatis mutandis to any other example unless specifically stated otherwise.

The present invention is not to be limited in scope by the specific examples described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying drawings.

KEY TO THE SEQUENCE LISTING

SEQ ID NO: 1: B subunit of the subtilase
cytotoxin (AB₅) of E. coli (SubB) wildtype amino
acid sequence.
AMAEWTGDARDGMFSGVVITQFHTGQIDNKPYFCIEGKQSAGSSISAC
SMKNSSVWGASFSTLYNQALYFYTTGQPVRIYYKPGVWTYPPFVKALT
SNALVGLSTCTTSTECFGPDRKKNS
SEQ ID NO: 2: SubBΔS106/ΔT107 mutant amino acid
sequence.
EWTGDARDGMFSGVVITQFHTGQIDNKPYFCIEGKQSAGSSISACSMK
NSSVWGASFSTLYNQALYFYTTGQPVRIYYKPGVWTYPPFVKALTSNA
LVGLSTCTTECFGPDRKKNS
SEQ ID NO: 3: Amino acid sequence of the glycan
binding motif of the native B subunit of AB₅.
TTSTE
SEQ ID NO: 4: SubB2M amino acid sequence
EWTGDARDGMFSGVVITQFHTGQIDNKPYFCIEGKQSAGSSISACSMK
NSSVWGASFSTLYNQALYFYTTGQPVRIYYEPGVWTYPPFVKALTSNA
LVGLSTCTTECFGPDRKKNS

### BRIEF DESCRIPTION OF THE DRAWINGS

FIG. 1: Neu5Gc levels in exosome samples derived from healthy individuals and cancer patients.

FIG. 2: Neu5Gc levels in exosome samples derived from healthy individuals and cancer patients controlling for exosome numbers.

FIG. 3: HPRT1 mRNA levels (mean CT following q-RT-PCR) following contact of plasma samples from lung cancer, prostate cancer and control patients with SubB2M and SubA12.

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., molecular biology, biochemistry, oncology and affinity based purification).

Unless otherwise indicated, the molecular and statistical techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

“N-glycolylneuraminic acid” or “Neu5Gc” are used herein to refer to particular glycans. In an example, the glycans terminate with alpha-2-3-linked N-glycolylneuraminic acid or alpha-2-6-linked N-glycolylneuraminic acid. Neu5Gc molecules are often referred to as sialic acid molecules. Sialic acids are α-keto acids with a nine-carbon backbone and are normally placed terminally in the reducing end of glycans. In an example, Neu5Gc can be defined by the following chemical formula, C₁₁H₁₉NO₁₀.

As shown in the diagram below, Neu5Gc is generated from NeuSAC by the enzyme CMP-N-acetylneuraminic acid hydroxylase (CMAH).

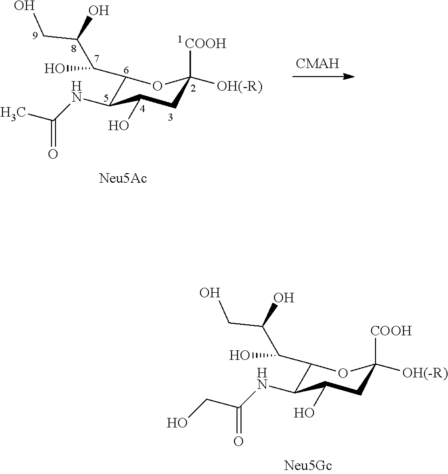

Neu5Gc is generally absent from normal human tissues because of deletion of an exon in CMAH. However, Neu5Gc has been detected on human cancer cells including cells from breast cancer, melanoma, malignant epithelial tumour, oesophageal carcinoma, gastric cancer, colorectal cancer, epidermoid carcinoma of rectum, pancreatic cancer, hepatocellular carcinoma, lymph node metastases, kidney cancer, urinary bladder cancer, ovarian cancer, uterine cancer, testicular cancer, prostate cancer, neuroblastoma, non-small cell lung cancer, lymphoma, neuroectodermal tumour (astrocytoma and glioblastoma), nephroblastoma (Wilms tumours), sarcoma, Ewing sarcomas and thyroid carcinoma (Labrada et al., Seminars in Oncology 2018; 45(1-2): 41-51).

The term “binding molecule” is used in the context of the present disclosure to refer to molecules that bind to Neu5Gc. In an example, binding molecules of the disclosure may be referred to as Neu5Gc-binding molecules. In an example, binding molecules of the disclosure bind to Neu5Gc expressed on the surface of tumour-derived extracellular vesicles. In an example, the binding molecule binds to the hydroxyl on the methyl group of the N-acetyl moiety that distinguishes Neu5Gc from Neu5AC (as shown in the above diagram). In another example, the binding molecule is “capable of binding alpha-2-3-linked N-glycolylneuraminic acid and alpha-2-6-linked N-glycolylneuraminic acid”. In an example, this means that the isolated molecule binds alpha-2-6-linked N-glycolylneuraminic acid glycans with substantially greater affinity than does a wild-type SubB protein (SEQ ID NO: 1; UniProtKB/Swiss-Prot: Q6EZC3.1), while also binding alpha-2-3-linked N-glycolylneuraminic acid glycans with a comparable affinity to that of a wild-type SubB protein (SEQ ID NO: 1). In an example, binding molecules of the disclosure bind to Neu5Gc or a sialyl linkage form thereof.

Exemplary binding molecules include immunoglobulin, antibodies, antigenic binding fragments and proteins such as the SubBΔS106/ΔT107 mutant (SEQ ID NO: 2) or variants thereof that bind to Neu5Gc (e.g. sequence variants of SEQ ID NO: 1; SEQ ID NO: 4). In an example, the binding molecule is a binding protein such as an antibody. In an example, the binding molecule is an aptamer. Other examples of binding molecules are discussed below. The term “immunoglobulin” will be understood to include any anti-Neu5Gc binding molecule comprising an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins.

The term “aptamer” or “aptamers” refers to non-naturally occurring nucleic acid or peptide structures which are folded into a three dimensional structure with high affinity for a target antigen, in this instance, Neu5Gc. These molecules are generally engineered through repeated rounds of in-vitro selection with a view to maximising target specificity.

The term “antibody” is used in the context of the present disclosure to refer to immunoglobulin molecules immunologically reactive with a particular antigen and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rlgG as discussed in Pierce Catalogue and Handbook, 1994-1995 (Pierce Chemical Co., Rockford, Ill.); Kuby, J., Immunology, 3^{rd} Ed., W. H. Freeman & Co., New York (1998). The term antibody also includes bivalent or bispecific molecules. Examples of bivalent and bispecific molecules are described in Kostelny et al. (1992) J Immunol 148:1547; Pack and Pluckthun (1992) Biochemistry 31:1579; Hollinger et al., 1993, supra, Gruber et al. (1994) J. Immunol.: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An “antigen binding fragment” of an antibody comprises one or more variable regions of an intact antibody. Examples of antibody fragments include Fab, Fab′, F(ab′)₂ and Fv fragments; diabodies; linear antibodies; single-chain antibody molecules and multispecific antibodies formed from antibody fragments. For example, the term antigen binding fragment may be used to refer to recombinant single chain Fv fragments (scFv) as well as divalent (di-scFv) and trivalent (tri-scFV) forms thereof.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that specifically binds to an antigen and, for example, includes amino acid sequences of CDRs; i.e., CDRl, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDRI, CDR2, and CDR3) refers to the amino acid residues of an antibody variable region the presence of which are major contributors to specific antigen binding. Each variable region typically has three CDR regions identified as CDRl, CDR2 and CDR3.

“Framework regions” (Syn. FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy C_{H}1, a linker, a C_{H}2 and a C_{H}3. In a heavy chain, a constant region comprises a Fc. In a light chain, a constant region generally comprise one constant domain (a CL1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of γ, α or δ heavy chain comprises two constant domains.

The term “naked” can be used to describe binding molecules of the present disclosure that are not conjugated to another compound or incorporated into a broader structure such as a molecular net disclosed herein. Put another way, the binding molecules of the present disclosure can be un-conjugated.

In contrast, the term “conjugated” can be used in the context of the present disclosure to describe binding molecules disclosed herein that are conjugated to another compound or structure, e.g., a Molecular Net or a detectable marker. Accordingly, in one example, the binding molecules of the present disclosure are “conjugated”. Binding molecules of the disclosure may be modified via conjugation or complexing with other chemical moieties, by post-translational modification (e.g. phosphorylation, ubiquitination, glycosylation), chemical modification (e.g. cross-linking, acetylation, biotinylation, oxidation or reduction) and/or conjugation with labels (e.g. fluorophores, enzymes, radioactive isotopes). Conjugated binding molecules of the disclosure retain their ability to bind Neu5Gc and sialyl linkage forms thereof, α2-3-linked N-glycolylneuraminic and α2-6-linked N-glycolylneuraminic. In an example, a binding molecule disclosed herein is conjugated to a detectable label such as a fluorescent label.

Reference to binding molecules generally should be taken to encompass both un-conjugated and conjugated forms thereof.

As used herein, the term “binds” refers to the interaction of a binding molecule with Neu5Gc and means that the interaction is dependent upon the presence of a particular structure (e.g., an antigenic determinant or epitope) on Neu5Gc. For example, a binding molecule of the disclosure recognizes and binds to a specific structural element of Neu5Gc rather than to molecules generally.

As used herein, the term “specifically binds” shall be taken to mean that the binding interaction between a binding molecule disclosed herein and Neu5Gc is dependent on detection of Neu5Gc by the binding molecule. Accordingly, the binding molecule preferentially binds or recognizes Neu5Gc even when present in a mixture of other molecules or organisms.

As used herein, the term, “capture” refers to the binding of anti-Neu5Gc binding molecule disclosed herein to tumour-derived extracellular vesicles. Terms such as “detect” or “detecting” are used to refer to processes and methods for detecting binding of anti-Neu5Gc binding molecule disclosed herein to tumour-derived extracellular vesicles.

As used herein, the term “isolate” or “isolating” or “isolation” refers to tumour-derived extracellular vesicles that have been separated from at least some components of a sample or methods of performing the same. These terms include gross physical separation of the tumour-derived extracellular vesicles from their natural environment (e.g. removal/purification from a sample obtained from a subject suspected of having cancer and/or removal/purification from a population of exosomes). The term “isolate” includes alteration of the tumour-derived extracellular vesicles relationship with other extracellular vesicles (e.g. non-cancerous extracellular vesicles) in a sample. For example, isolating tumour-derived extracellular vesicles from a heterogeneous extracellular vesicle population can provide a pure or partially pure population of tumour-derived extracellular vesicles. In an example, “isolating” according to the present disclosure increases the ratio of tumour-derived extracellular vesicles: non-cancerous extracellular vesicles in a sample. Tumour-derived extracellular vesicles bound by a Neu5Gc binding molecule disclosed herein can be isolated from a sample using various methods. For example, binding molecules of the disclosure which are bound to Neu5Gc on the surface of tumour-derived extracellular vesicles are separated from the sample. In an example, affinity based separation methods are used. Such methods leverage the affinity of a binding molecule for Neu5Gc to isolate tumour-derived extracellular vesicles expressing Neu5Gc on their surface from a sample. In another example, Neu5Gc binding molecules disclosed herein can be used to tag or label tumour-derived extracellular vesicles so that labelled or tagged extracellular vesicles can be filtered or sorted from a sample. For example, a fluorescence based sorting system may be applied to a sample, selecting for tumour-derived extracellular vesicles labelled with a binding molecule disclosed herein.

As used herein, the term “extracellular vesicles” refer to a heterogeneous group of membranous structures derived from outward budding of the plasma membrane or endosomal system of cells. Extracellular vesicles can comprise small extracellular vesicles, exosomes and microvesicles. The extracellular vesicles derived from tumour cells and are referred to as “tumour-derived extracellular vesicles” herein. Such extracellular vesicles are generally secreted from tumour cells into their surrounding micro-environment. Extracellular vesicles comprise small extracellular vesicles (50-200 nm), microvesicles (0.2-1 μm), exomeres and exosomes (30-150 nm), and oncosomes (1 μm up to 10 μm). Exomeres are membrane bound nanoparticles. In one example, the tumour-derived extracellular vesicles are tumour-derived small extracellular vesicles (50-200 nm), microvesicles (0.2-1 μm) or exosomes (30-150 nm). In another example, the tumour-derived extracellular vesicles are tumour-derived exosomes. In another example, the tumour-derived extracellular vesicles are exomeres. In an example, the tumour-derived exosomes are also CD63+.

As used herein, the term “expressing” refers to extracellular vesicles that display a protein (e.g. Neu5Gc), for example, on their surface. For example, extracellular vesicles express membrane associated proteins. In an example, tumour-derived extracellular vesicles isolated according to the present disclosure express Neu5Gc on their surface in a form accessible for binding by an anti-Neu5Gc binding molecule disclosed herein.

As used herein, the term “subject” refers to a human subject. In an example, the subject is suspected of having cancer. In another example, the subject has been diagnosed with cancer. In this example, the subject has previously been diagnosed with cancer and is in remission. In an example, the subject is enrolled in a screening program on the basis of their prior cancer diagnosis.

As used herein, the term “fragment” refers to a portion of a binding molecule disclosed herein which maintains a defined activity of the full-length binding molecule, specifically the ability to bind Neu5Gc. In one example, the fragment has at least 50%, 55%, 60%, 65%, 70%, 75%, 80%, 85%, 90%, 91%, 92%, 93%, 94%, 95%, 96%, 97%, 98% or 99% of the ability of SEQ ID NO: 1 or SEQ ID NO: 2 to bind a2-3-linked N-glycolylneuraminic and α2-6-linked N-glycolylneuraminic. In an example, the fragment is derived from SEQ ID NO: 1, SubBAS106/AT107 mutant (SEQ ID NO: 2) or SEQ ID NO: 4. In an example, the fragment derived from SEQ ID NO: 1 has an amino acid sequence which comprises SEQ ID NO: 1 or SEQ ID NO: 4.

In an example, the Neu5Gc binding molecule is a variant derived from SEQ ID NO: 1 or either SEQ ID NO: 2 or SEQ ID NO: 4. In an example, the variant comprises a sequence modification to SEQ ID NO: 3. As used herein, the term “variant” refers to a binding molecule with a difference(s) in one or more amino acid sequence(s) to a binding molecule disclosed herein such as SEQ ID NO: 1 or SEQ ID NO: 2 but retains the ability to bind α2-3-linked and α2-6-linked Neu5Gc. For example, a variant of SEQ ID NO: 2 can comprise the amino acid sequence shown in SEQ ID NO: 4. In one example, the variant has at least 50%, 55%, 60%, 65%, 70%, 75%, 80%, 85%, 90%, 91%, 92%, 93%, 94%, 95%, 96%, 97%, 98% or 99% of the ability of a binding molecule having an amino acid sequence as shown in SEQ ID NO: 1, SEQ ID NO: 2 or SEQ ID NO: 4 to bind α2-3-linked N-glycolylneuraminic and α2-6-linked N-glycolylneuraminic. In one example, the variant shares at least 80%, 81%, 82%, 83%, 84%, 85%, 86%, 87%, 88%, 89%, 90%, 91%, 92%, 93%, 94%, 95%, 96%, 97%, 98%, 99% sequence identity with an amino acid sequence of SEQ ID NO: 1, SEQ ID NO: 2 or SEQ ID NO: 4. In an example, the sequence identity is determined over at least 50, 60, 70, 80, 90, 100 amino acids of the reference sequence (e.g., SEQ ID NO: 1). The variant disclosed herein may have one or more amino acids deleted or substituted by different amino acids.

In an example, the binding molecule is at least 10, 20, 50, 60, 70, 80, 90, 100amino acids long. In another example, the binding molecule is at least 50, 60, 70, 80, 90, 100 amino acids long. In another example, the binding molecule is at least 50 amino acids long. In an example, the variant or fragment comprises an amino acid sequence of SEQ ID NO: 1, SEQ ID NO: 2 or SEQ ID NO: 4 that is at least 10, 20, 50, 60, 70, 80, 90, 100 amino acids long. In another example, the variant or fragment comprises an amino acid sequence of SEQ ID NO: 1, SEQ ID NO: 2 or SEQ ID NO: 4 that is at least 50, 60, 70, 80, 90, 100 amino acids long. In another example, the variant or fragment comprises an amino acid sequence of SEQ ID NO: 1, SEQ ID NO: 2 or SEQ ID NO: 4 that is at least 50 amino acids long. In an example, the variant or fragment comprises SEQ ID NO: 3. In an example, the variant or fragment comprises a modification to SEQ ID NO: 3.

As used herein, the term “SubB” refers to a subtilase cytotoxin B subunit protein of bacterial AB5 toxins (see e.g. WO2018/085888; SEQ ID NO: 1). SubB has the ability to bind α2-3-linked N-glycolylneuraminic acid. In an example, binding molecules of the disclosure encompass variants of SubB that have one or more amino acid residues of the amino acid sequence TTSTE (SEQ ID NO: 3) modified. Exemplary modifications include substitutions, deletions and additions. In an example, the modification is a substitution or a deletion. In an example, the modification is a deletion.

As used herein, the term “SubBAS106/AT107 mutant” refers to a mutant form of mature SubB having an amino acid sequence as shown in SEQ ID NO: 2. SubBΔS106/ΔT107 mutant has the ability to bind to two sialyl linkage forms of Neu5Gc (α2-6-linked N-glycolylneuraminic acid and α2-3-linked N-glycolylneuraminic acid). Another exemplary mutant form of mature SubB comprises the amino acid sequence set forth in SEQ ID NO: 4. SEQ ID NO: 4 substantially corresponds with SEQ ID NO: 2 but for an amino acid modification at potion 79 (K79E). Both mutant forms of mature SubB have the ability to bind to two sialyl linkage forms of Neu5Gc (a2-6-linked N-glycolylneuraminic acid and α2-3-linked N-glycolylneuraminic acid).

As used herein, the term “layered, multipolymeric molecular net structure” refers to a covalently linked multi-layered three-dimensional matrix comprising a binding molecule that binds Neu5Gc or a sialyl linkage form thereof such as α2-3-linked N-glycolylneuraminic acid or α2-6-linked N-glycolylneuraminic acid, and linkers and spacers. In an example, the placement and spacing of the Neu5Gc binding molecule, linkers and spacers of the layered, multipolymeric molecular net structure confers a density of Neu5Gc-binding molecules within each layer and a porosity of the net structure such that the net structure may also allow size exclusion. The layered, multipolymeric molecular net structure can be applied to any solid surface (e.g. magnetic beads, dipsticks, ELISA plate wells). Net structures and components thereof are discussed further below. In an example, the layered multipolymeric molecular net structure comprises a binding molecule that binds α2-3-linked N-glycolylneuraminic acid and α2-6-linked N-glycolylneuraminic acid. In an example, the layered multipolymeric molecular net structure comprises at least two layers. In another example, the layered multipolymeric molecular net structure comprises at least three layers. In another example, the layered multipolymeric molecular net structure comprises three layers.

As used in this specification and the appended claims, terms in the singular and the singular forms “a,” “an” and “the,” for example, optionally include plural referents unless the content clearly dictates otherwise.

As used herein, the term “about”, unless stated to the contrary, refers to +/−10%, more preferably +/−5%, more preferably +/−1%, of the designated value.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Binding Molecules

Binding molecules suitable for use in the present disclosure are not particularly limited so long as they can bind to Neu5Gc or a sialyl linkage form thereof expressed on the surface of tumour-derived extracellular vesicles. In an example, the binding molecule binds to α2-6-linked N-glycolylneuraminic acid and α2-3-linked N-glycolylneuraminic acid. Terms such as “anti-Neu5Gc binding molecule” and “Neu5Gc binding molecule” are used herein to refer to molecules that bind Neu5Gc.

In an example, the binding molecule is derived from the pentameric B sub unit of subtilase cytotoxin (SubAB) from Escherichia coli (SEQ ID NO: 1; see for example binding proteins described in Day et al. (2017) Scientific Reports., 7:1495). For example, the binding protein can be the mutant SubBΔS106/ΔT107 as described by Day et al. (2017). Other examples of Neu5Gc binding molecules are described in Wang et al. (2018) Biochem Biophys Res Comm., 500:765-771.

In one example, the binding molecule is a mutant Subtilase cytotoxin B subunit protein (SubBΔS106/ΔT107) having the ability to bind to two sialyl linkage forms of Neu5Gc (α2-3-linked N-glycolylneuraminic acid and α2-6-linked N-glycolylneuraminic acid). In an example, the binding molecule has an amino acid sequence as shown in SEQ ID NO: 2. In an example, the binding molecule has an amino acid sequence as shown in SEQ ID NO: 4.

In one example, the binding molecule comprises the amino acid sequence of SubB or a variant thereof, wherein one or more amino acid residues of the binding molecules amino acid sequence TTSTE (SEQ ID NO:3) are modified and, wherein the binding molecule is capable of binding α2-3-linked N-glycolylneuraminic acid and α2-6-linked N-glycolylneuraminic acid.

In one example, binding molecule is a variant of SEQ ID NO: 1 which comprises a non-conservative substitution or deletion of at least one of the underlined residues of TTSTE. In another example, the binding molecule comprises a deletion of the underlined residue of TTSTE. In another example, the binding molecule comprises deletion of the underlined residues of a TTSTE. In another a example, the binding molecule comprises deletion of the underlined residues of TTSTE.

In an example, the amino acid sequence of the binding molecule consists of the amino acid sequence of SEQ ID NO: 2. In an example, the amino acid sequence of the binding molecule consists of the amino acid sequence of SEQ ID NO: 4.

In an example, the binding molecule is a bacterial-derived binding protein that binds to Neu5Gc. In an example, the bacterial-derived binding protein is selected by a sequence search which selects for proteins having a sequence which binds to Neu5Gc (e.g. SEQ ID NO: 1, SEQ ID NO: 2, SEQ ID NO: 3 or SEQ ID NO: 4). Suitable methods for identifying bacterial proteins in this way include a protein blast search (Blastp; Altschul et al. (1990) J. Mol. Biol., 215:403-410) and would be known to those of skill in the art.

In one example, the binding molecule comprises:
(i) a single chain Fv fragment (scFv);
(ii) a dimeric scFv (di-scFv); or
(iii) a diabody;
(iv) a triabody;
(v) a tetrabody;
(vi) a nanobody;
(vii) a Fab;
(viii) a F(ab′)₂;
(ix) a Fv;
(x) an aptamer;
(xi) one of (i) to (ix) linked to a constant region of an antibody, Fc or a heavy chain constant domain (C_{H}) 2 and/or C_{H}3;
(xii) one of (i) to (ix) linked to albumin or a functional fragment or variants thereof or a protein that binds to albumin; or
(xiii) an antibody that binds to Neu5Gc. In this example, the binding molecule may compete with a binding molecule comprising an amino acid sequence as shown in SEQ ID NO: 2 or SEQ ID NO: 4 for binding to Neu5Gc.

In one example, the binding molecule is an antibody. Exemplary antibodies are full length and/or naked antibodies.

In one example, the binding molecule is an anti-Neu5Gc antibody. In an example, the anti-Neu5Gc antibody is produced in chickens. An example of a binding molecule is polyclonal chicken anti-Neu5Gc antibody (e.g. Creative Diagnostics; Biolegend). In one example, the anti-Neu5Gc antibody is a monoclonal anti-Neu5Gc antibody.

In one example, the binding molecule is recombinant, chimeric, CDR grafted, humanized, synhumanized, primatized, deimmunized or human.

In one example, the binding molecule is a DNA or RNA aptamer.

In an example, the binding molecule is identified by screening for binding molecules which bind to Neu5Gc. In another example, the binding molecule is identified by screening for antibodies that compete with SubBΔS106/ΔT107 for binding to Neu5Gc.

In an example, the Neu5Gc binding molecule is detectably labelled. For example, the Neu5Gc binding molecule can be fluorescently labelled.

### Molecular Nets Comprising Binding Molecule(s)

The present disclosure also provides a method of detecting and/or isolating tumour-derived extracellular vesicles from a sample, the method comprising:
contacting the sample with a layered, multipolymeric molecular net structure having multiple layers of at least one binding molecule that binds Neu5Gc under conditions that enable binding of the binding molecule to tumour-derived extracellular vesicles in the sample; and isolating the layered, multipolymeric molecular net structure from the sample.

The present disclosure further provides a method of isolating tumour-derived extracellular vesicles from a sample, the method comprising:
contacting the sample with a layered, multipolymeric molecular net which comprises multiple layers of at least one binding molecule that binds exosomes under conditions that enable binding of the molecule to tumour-derived extracellular vesicles in a sample; and isolating the layered, multipolymeric molecular net from a sample to enable detection of Neu5Gc on isolated tumour-derived extracellular vesicles, wherein, Neu5Gc can be detected using a Neu5Gc binding molecule.

In an example, Neu5Gc is detected using an antibody directed against Neu5Gc or SubBΔS106/ΔT107 mutant.

In an example, the molecular net has a pseudorandom structure. In an example, the layered, multipolymeric molecular net structure can further comprise one or more binding molecule(s) that bind to Neu5Gc on extracellular vesicles such as exosomes.

Methods of making layered, multipolymeric molecular net structures disclosed herein are known in the art (see for example, WO2011/066449; WO2014011673; WO2014153262) and/or can be prepared by a method described herein. A brief overview of a suitable method is also exemplified below.

In an example, the layered, multipolymeric molecular net structure may be built in a layered or striated manner. For example, a solution containing a homogenous or heterogeneous mixture of one or more binding molecule(s) is deposited at a site and a crosslinker(s) is added to the solution under conditions in which the crosslinker(s) and binding molecule(s) form a cross-linked net. In an example, the cross-linker is added with stirring. The binding molecule(s) may, for example, be deposited on a planar surface (e.g. carbon, polymeric surface or glass surface). Another exemplary surface is a bead such as a magnetic bead. This may be followed by addition of homogenous or heterogeneous mixtures of one or more binding molecule(s), followed by additional crosslinker(s). In an example, binding molecule(s) and crosslinker(s) are premixed before being incorporated into a molecular net structure. In an example, each layer of the molecular net comprises a Neu5Gc binding molecule. In an example, each layer of the molecular net is prepared by pre-mixing a binding molecule(s) and crosslinker(s). The result may be a branched pseudorandom copolymer comprising one or more binding molecule(s) and crosslinkers. Examples of crosslinkers are known in the art and include BS3, [N-e-Maleimidocapropyl]succinimide ester (EMCS), ethylene glycol bis[succinimidylsuccinate] (EGS), NHS-(PEG)n-maleamide, NHS-(PEG)n-NHS, and where n can be 1 to 50. In an example, the chemical crosslinkers can be between 2 and 200 Angstroms in length.

In an example, the layers of the molecular net comprise antibodies that bind surface epitopes of exosomes generally. In an example, these nets are used to purify exosomes from a sample before detecting expression of Neu5Gc on exosomes using a binding molecule disclosed herein such as SubBΔS106/ΔT107. Examples of such nets include nets comprising an anti-CD63 binding protein such as an anti-CD63 antibody.

### Sample Preparation

The methods of the present disclosure can be performed on various samples. As used herein, the term “sample” may be a sample obtained from a subject or a cell culture sample. For example, the sample can be a bodily fluid of the subject. In one example, the sample is selected from a group consisting of blood, serum, plasma, urine, saliva, faeces, tears, broncho-alveolar lavage fluid (BALF), cerebrospinal fluid (CSF) and seminal fluid. In one example, the sample is blood. In another example, the sample is a cell culture or supernatant thereof. In another example, the sample is a cell culture comprising tumour cells or supernatant thereof. In another example, the sample selected from a group consisting of plasma and serum. In an example, the sample is a serum sample. In an example, the sample is a population of exosomes. In an example, the sample is a population of exosomes obtained from a patient suspected of having cancer. In an example, the sample is purified or partially purified before detecting/isolating tumour-derived extracellular vesicles according to the present disclosure. For example, a serum sample may be purified to remove cells. In an example, other components (e.g. debris, albumin or free Neu5Gc) originally within the sample are removed or partially removed from the sample before performing the methods of the present disclosure. In an example, the sample is the supernatant of a tissue culture. In an example, the sample is substantially free of cells.

The sample includes extracts, derivatives, fractions or suspensions of an original sample obtained from a subject or cell culture disclosed herein.

In one example, the sample is a purified or partially purified population of extracellular vesicles. In one example, the sample is a purified population of exosomes.

In one example, the sample is a partially purified population of exosomes. In an example, these samples are purified to remove non-extracellular vesicle bound Neu5Gc.

Methods of isolating a heterogeneous population of extracellular vesicles are known in the art (e.g. WO2010121335; WO2013188832; WO2014159662). In other examples, a heterogeneous population of extracellular vesicles can be purified from a sample using ultracentrifugation, filtration or column chromatography. In an example, extracellular vesicles can be purified from a sample using one or more protein(s) associated with the membrane of the extracellular vesicles. For example, affinity chromatography targeting one or more protein(s) associated with the membrane of extracellular vesicles can be used.

In one example, a population of extracellular vesicles such as exosomes is purified based on expression of one or more protein(s) selected from a group consisting of CD63, b2microglobulin, CD11, CD81, CD13, EGFR. In an example, the extracellular vesicles are purified based on expression of one or more of the above referenced proteins before being subject to a method of the disclosure to detect/isolate tumour-derived extracellular vesicles.

### Isolation and/or Detection of Tumour-Derived Extracellular Vesicles

The present inventors identified that Neu5Gc is associated with the external membrane of tumour-derived extracellular vesicles. These findings provided a basis for detecting, capturing, labelling and/or isolating tumour-derived extracellular vesicles from a sample based on their expression of Neu5Gc. Accordingly, in an example, the present disclosure provides a method of isolating tumour-derived extracellular vesicles from a sample, the method comprising:
contacting a sample comprising tumour-derived extracellular vesicles with a binding molecule that binds to Neu5Gc under conditions that enable binding of the binding molecule to the tumour-derived extracellular vesicles in the sample;
isolating the binding molecule that has bound to tumour-derived extracellular vesicles from the sample.

In one example, the method further comprises disassociating bound tumour-derived extracellular vesicles from the isolated binding molecule(s). In an example, tumour-derived extracellular vesicles are disassociated from the binding molecule using a buffer that can disrupt the binding of the tumour-derived extracellular vesicles to the binding molecule. Exemplary disassociation buffers are discussed below.

In other examples, tumour-derived extracellular vesicles such as exosomes can be captured from various samples using binding molecules disclosed herein. In an example, such methods comprise contacting a sample comprising tumour-derived extracellular vesicles with a binding molecule that binds to N-glycolylneuraminic acid (Neu5Gc) under conditions that enable binding of the binding molecule to the tumour-derived extracellular vesicles in the sample; and, optionally, isolating the binding molecule from the sample. In an example, tumour-derived extracellular vesicles bound or labelled by a Neu5Gc binding molecule disclosed herein can be isolated. For example, the Neu5Gc binding molecule can be used to detect rather than isolate, wherein another suitable isolation method is employed to capture/isolate tumour-derived extracellular vesicles expressing Neu5Gc on their membrane surface. For example, a Neu5Gc binding protein can be used to label a population of tumour-derived extracellular vesicles in a sample before the labelled extracellular vesicles are captured/isolated. In an example, the sample is a purified population of exosomes, wherein the tumour-derived exosomes are detectably labelled with a Neu5Gc binding protein disclosed herein, for example, a fluorescently labelled Neu5Gc binding protein. In this example, the tumour-derived exosomes can be subsequently isolated on the basis of the detectable label (e.g. fluorescent sorting).

In another example, Neu5Gc binding molecules described herein are used in affinity based purification of tumour-derived extracellular vesicles expressing Neu5Gc on their membrane surface. For example, an anti-Neu5Gc binding molecule described herein can be attached to a solid support or matrix. In another example, a Neu5Gc binding protein is incorporated into a molecular net disclosed herein.

In an example, the methods of the disclosure comprise contacting a sample comprising tumour-derived extracellular vesicles with an anti-Neu5Gc binding molecule under conditions that enable binding of the binding molecule to the tumour-derived extracellular vesicles in the sample; and, optionally, detecting and/or isolating the tumour-derived extracellular vesicles.

Isolated exosomes can subsequently be analysed as described herein.

In examples disclosed herein, binding of the binding molecule to tumour-derived extracellular vesicles can be detected to indicate the presence of tumour-derived exosomes in a sample by various means (visualisation, chemical and immunochemical analysis). Such detection indicates the presence of tumour-derived extracellular vesicles in the sample. In such examples, biochemical analysis of tumour-derived extracellular vesicles is possible without the need to elute the tumour-derived extracellular vesicles from the binding molecule.

In an example, the method further comprises isolating tumour-derived extracellular vesicles.

Methods of “detecting” binding are not particularly limited so long as they can detect binding between binding molecules disclosed herein and Neu5Gc expressed on the surface of tumour-derived extracellular vesicles. Examples include high resolution microscopy, such as electron microscopy or confocal microscopy in which binding molecule/extracellular vesicle complex aggregates are detected and immunosorbent assays, which use a tagged antibody to allow the level of bound exosomes to be detected, and, optionally quantified. Examples of methods for detecting binding disclosed herein may be assisted by use of a detectably labelled anti-Neu5Gc binding molecule (e.g. fluorescent label).

In an example, the number of tumour-derived extracellular vesicles in a sample labelled with a Neu5Gc binding protein is quantified. Examples of appropriate quantification methods are dependent on the detectable label used. For example, a fluorescently labelled exosomes may be counted.

To detect and/or isolate tumour-derived extracellular vesicles according to the disclosure, a sample is contacted with a binding molecule disclosed herein. It is considered that terms such as “contacting”, “exposing” or “applying” are terms that can, in context, be used interchangeably in the present disclosure. The term contacting, requires that the binding molecule be brought into contact with a sample to detect whether Neu5Gc is expressed on the surface of extracellular vesicles in the sample. Binding indicates that extracellular vesicles expressing Neu5Gc are present in the sample. Binding may be detected using various binding molecule/antigen binding detection techniques known in the art. For example, an immunoassay incorporating a binding molecule disclosed herein may be used. In an example, binding is detected using Surface Plasmon Resonance (SPR). In an example, extracellular vesicles in the sample may be labelled before performing the methods of the present disclosure. For example, exosomes can be fluorescently labelled.

It is envisaged that determining conditions that enable binding of a binding molecule disclosed herein to tumour-derived extracellular vesicles expressing Neu5Gc would be well within the purview of those skilled in the art. For example, appropriate concentrations of binding molecule can be determined using the examples below as a starting point. In general, binding molecules of the disclosure can be provided in a suitable solution and concentration so that they may recognise and bind to Neu5Gc on the surface of tumour-derived extracellular vesicles.

In an example, a sample comprising extracellular vesicles is obtained from a subject. The sample is optionally purified to isolate a population of extracellular vesicles from the sample. The extracellular vesicles in the sample are contacted with a Neu5Gc binding molecule under conditions that enable binding of the binding molecule to tumour-derived extracellular vesicles expressing Neu5Gc. Tumour-derived extracellular vesicles bound by the Neu5Gc binding protein are then isolated from the sample.

In another example, a sample comprising extracellular vesicles is obtained from a subject. The sample is optionally purified to isolate a population of extracellular vesicles from the sample. The sample is passed through an affinity column comprising one or more binding molecule(s) that binds Neu5Gc. The affinity column is washed before the Neu5Gc expressing extracellular vesicles are eluted from the affinity column.

In another example, a sample comprising extracellular vesicles is obtained from a subject. The sample is optionally purified to isolate a population of extracellular vesicles from the sample. The sample is incubated for a period of time (e.g. 30 minutes) with one or more binding molecule(s) that bind Neu5Gc in a fixed volume. Binding molecule bound to tumour-derived extracellular vesicles expressing Neu5Gc is subsequently detected and/or isolated from the fixed volume (e.g. using a magnetic holder where the binding molecule is fixed to a magnetic bead). Optionally, detected binding is quantified to provide a measure of tumour-derived extracellular vesicles in the sample. In this instance, it may be preferable to provide a labelled binding molecule and/or label extracellular vesicles (e.g. fluorescently labelled) in the sample.

In an example, the method described herein further comprises dissociating bound tumour-derived extracellular vesicles from the binding molecule and collecting the dissociated tumour-derived extracellular vesicles. Various methods of dissociating bound tumour-derived extracellular vesicles from binding molecules disclosed herein are known by those skilled in the art. For example, a suitable dissociation buffer wash can be implemented in the methods of the disclosure such as, for example, dissociation buffers described in Ishida et al. (2020) Sci Rep 10., 18718. Alternatively, in an example, the dissociation buffer is phosphate buffered saline (PBS). In other examples, affinity chromatography is used to isolate tumour-derived extracellular vesicles from samples disclosed herein.

### Analysis of Tumour-Derived Extracellular Vesicle

In one example, the method described herein further comprises analysing isolated tumour-derived extracellular vesicles such as exosomes. In an example, the contents of the extracellular vesicles are analysed. In an example, the protein expression profile is analysed. In another example, the microRNA expression profile is analysed.

Tumour-derived extracellular vesicles isolated according to the present disclosure can be analysed using various methods known in the art. For example, analysis can include quantifying the number and/or composition (protein, nucleic acids, lipids or sugar content) of tumour-derived extracellular vesicles. For example, nucleic acids and/or proteins may be isolated from tumour-derived extracellular vesicles and analysed (e.g. quantified). Exemplary methods used for such analysis include quantitative amplification reactions such as PCR, DNA and/or RNA sequence analysis, small RNA sequencing, microRNA sequencing, quantitative protein expression analysis, ELISA, mass spectrometry, immunoaffinity capture, cytometric analysis, Fourier transform Infra Red Spectroscopy (FTIR) and lectin binding.

In an example, the analysed extracellular vesicles are selected from a group consisting of small extracellular vesicles, exomeres, exosomes and microvesicles. In another example, the analysed extracellular vesicles are small extracellular vesicles. In another example, the analysed extracellular vesicles are microvesicles. In another example, the analysed extracellular vesicles are exosomes.

### Tumour Detection

It is envisaged that methods of detecting tumour-derived extracellular vesicles such as exosomes disclosed herein can be used to determine whether a subject has cancer. In an example, such methods can be used to detect cancer in a subject with symptoms that are indicative of cancer. In an example, the methods disclosed herein can be used in routine cancer screening. For example samples can be obtained from subjects enrolled in routine cancer screening and the samples can be assessed for the presence of cancer derived extracellular vesicles using the methods disclosed herein. In an example, after detecting the presence of tumour-derived extracellular vesicles using a method disclosed herein, the location and type of cancer can be confirmed via further screening of the subject (e.g. PET-scan, biopsy, cytology, histology).

Types of cancer detected according to the disclosure are not particularly limited so long as they secrete tumour-derived extracellular vesicles such as exosomes that express Neu5Gc. In an example, the cancer is selected from a group consisting of breast cancer, melanoma, malignant epithelial tumour, oesophageal carcinoma, gastric cancer, colorectal cancer, epidermoid carcinoma of rectum, pancreatic cancer, hepatocellular carcinoma, lymph node metastases, kidney cancer, urinary bladder cancer, ovarian cancer, uterine cancer, testicular cancer, prostate cancer, neuroblastoma, non-small cell lung cancer, lymphoma, neuroectodermal tumour (astrocytoma and glioblastoma), nephroblastoma (Wilms tumours), sarcoma, Ewing sarcomas and thyroid carcinoma. In an example, the cancer is breast cancer. In an example, the cancer is breast, ovarian or pancreatic cancer. In an example, the cancer is a solid tumour. In an example, the cancer is breast cancer. In another example, the cancer is lung cancer. In another example, the cancer is prostate cancer. In another example, the cancer is breast, lung or prostate cancer.

### Neu5Gc Tumour-Derived Extracellular Vesicle Population or Composition

The present disclosure also provides a population of tumour-derived extracellular vesicles enriched for extracellular vesicles expressing Neu5Gc. In an example, the population of tumour-derived extracellular vesicles are obtained by contacting a sample of extracellular vesicles with a binding molecule disclosed herein. In an example, the tumour-derived extracellular vesicles are provided in a composition. In an example, the composition comprises a binding molecule which comprises SEQ ID NO: 4.

The present disclosure further provides a composition comprising extracellular vesicles enriched for extracellular vesicles expressing N-glycolylneuraminic acid (Neu5Gc), wherein the extracellular vesicles are tumour-derived extracellular vesicles isolated/purified by a method described herein. In an example, an enriched sample has a greater ratio of tumour-derived extracellular vesicles: to non-tumour-derived extracellular vesicles. In an example, the increased ratio is determined relative to the ratio of a starting sample that has not been subject to a method disclosed herein.

In one example, at least 20% of the extracellular vesicles in the composition or the population express Neu5Gc. In another example, at least 30% of the extracellular vesicles in the composition or the population express Neu5Gc. In another example, at least 40% of the extracellular vesicles in the composition or the population express Neu5Gc. In another example, at least 50% of the extracellular vesicles in the composition or the population express Neu5Gc. In another example, between 15% and 50% of the extracellular vesicles in the composition or the population express Neu5Gc.

In another example, between 30% and 50% of the extracellular vesicles in the composition or the population express Neu5Gc.

In another example, more than 60%, more than 70% of the extracellular vesicles in the composition or the population are extracellular vesicles expressing Neu5Gc.

In an example, the present disclosure encompasses a binding molecule that binds to Neu5Gc when used in a method defined herein such as detecting tumour-derived extracellular vesicles.

In an example, the present disclosure encompasses a binding molecule that binds to N-glycolyneuraminic acid (Neu5Gc) when used for capturing and/or detecting tumour-derived extracellular vesicles in a sample. In an example, the binding molecule comprises an amino acid sequence as shown in SEQ ID NO: 4.

### Methods of Screening

In an example, the methods of the present disclosure relate to methods of screening for biomarkers of tumour derived extracellular vesicles, such as exosomes. In an example, the method comprises capturing tumour derived extracellular vesicles from a sample according to a method described herein, the method further comprising screening the captured tumour derived extracellular vesicle(s) for biomarker(s). In an example, the biomarker(s) further characterise the tumour derived extracellular vesicles. In another example, the biomarker(s) distinguish the tumour derived extracellular vesicles from normal tumour derived extracellular vesicles. In an example, screening the captured tumour derived extracellular vesicle(s) for biomarker(s) involves a DNA (exoDNA) and/or RNA based assessment using, for example, quantitative reverse transcription PCR. In an example, screening the captured tumour derived extracellular vesicle(s) for biomarker(s) involves a protein based assessment using, for example, mass spectrometry. Biomarker(s) identified by these screening methods may then be incorporated into the methods of the present disclosure to capture/detect tumour derived extracellular vesicles.

### EXAMPLES

### Example 1: Neu5Gc is Expressed on the External Membrane of Tumour-Derived Exosomes

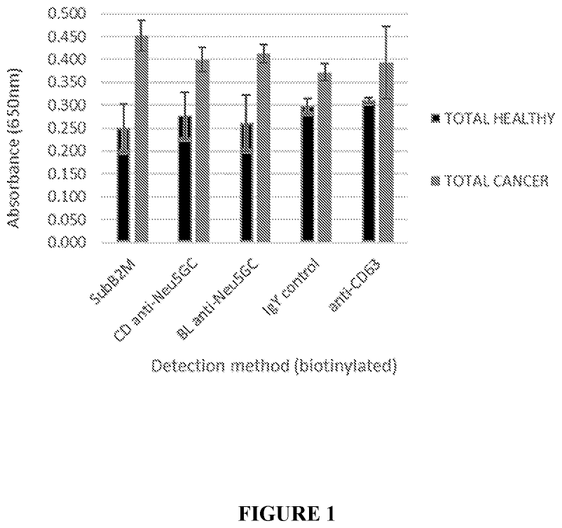

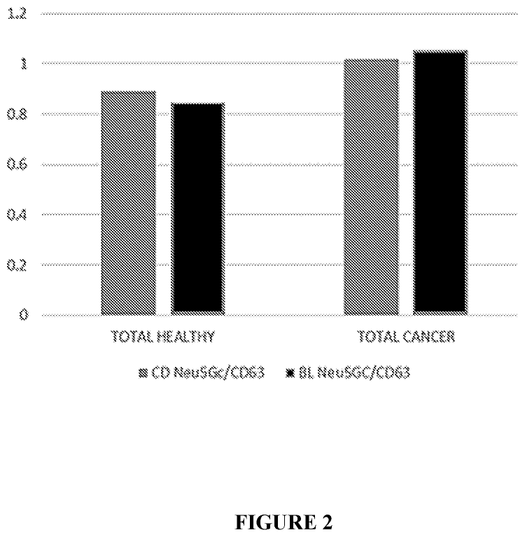

Binding assays were performed to determine whether Neu5Gc could be detected on the membrane surface of tumour-derived exosomes. Exosome populations derived from healthy individuals and cancer patients were contacted with a selection of biotinylated Neu5Gc binding molecules including SubBΔS106/ΔT107 and two anti-Neu5Gc antibodies (BioLegend [BL]; Creative Diagnostics [CD]). Samples were also contacted with IgY to provide a control for non-specific binding and an anti-CD63 antibody (marker specific to exosomes) to control for exosome numbers in each sample. FIG. 1 surprisingly shows that exosomes derived from cancer subjects express Neu5Gc on their membrane surface in a form detectable by all Neu5Gc binding molecules tested. CD and BL anti-Neu5Gc antibodies and SubBΔS106/ΔT107 all detected Neu5Gc positive exosomes in exosome populations derived from cancer samples as shown by elevated Neu5Gc levels relative to IgY control. In contrast, the Neu5Gc binding molecules did not detect Neu5Gc levels above control in the exosome populations derived from heathy individuals (FIG. 1). Exosomes were present in all samples assessed as shown by the levels of CD63 detected (FIG. 1). FIG. 2 controls for exosome number.

These data suggest that Neu5Gc is expressed on the membrane surface of tumour-derived exosomes and that tumour-derived exosomes can be detected on this basis using various anti-Neu5Gc binding molecules. These data also suggest that tumour-derived exosomes can be purified from a population of exosomes based on expression of Neu5Gc.

### Example 2: SubBΔS106/ΔT107 Molecular Net Development

A layered, multipolymeric molecular net structure having multiple layers of SubBΔS106/ΔT107 that binds to Neu5Gc (“SubB2M molecular net”) was produced by covalently linking SubBΔS106/ΔT107 using linkers and spacers on a magnetic bead.

In brief, 0.1-0.5 mg/ml SubBΔS106/ΔT107 was prepared before adding linker (BS3, BS(PEG)9, BS(PEG)7, BS(PEG)5, BS2G-d0, BS2G-d4; Tri Link) with stirring and then incubating at room temperature in the dark to provide a first net layer. The first net layer was covalently linked to a magnetic bead. A second net layer was then produced by adding linker to SubBΔS106/ΔT107 solution with stirring followed by incubation at room temperature in the dark. A third net layer was then produced by adding linker to SubBΔS106/ΔT107 solution with stirring followed by incubation at room temperature in the dark.

### Example 3: Neu5Gc Based Purification of Tumour-Derived Exosomes

Binding assays were performed to determine whether tumour-derived exosomes could be purified on the basis of Neu5Gc expression. MCF7 exosomes (1×10⁷ exosomes) were pre-labelled with Exo-Red (SBI) in 1 mL of Dulbecco's Phosphate Buffered Saline (DPBS). SubBΔS106/ΔT107 molecular nets, produced from the method as described in Example 2, were incubated with the pre-labeled MCF7 exosomes for 30 minutes. The incubation mixture was washed three times with 1 mL of DPBS. The mean fluorescent intensity (MFI) of fluorescently labelled MCF7 exosomes was then determined in the flow through (FT), wash and bound fractions (WABF) of the binding assays (see Tables 1 and 2).

Table 1 shows the results of contacting a fixed amount of MCF7 exosomes in a sample with varying amounts of SubBΔS106/ΔT107 molecular net. In the presence of 15μL SubBΔS106/ΔT107 molecular net, approximately 49% of MCF7 exosomes were detected in the WABF compared to total MFI fraction of the binding assay, which was approximately 3 times the amount of the control (absence of SubBΔS106/ΔT107 molecular net) (Table 1). These data show that Neu5Gc is expressed on the membrane surface of tumour-derived exosomes and that Neu5Gc can be used to detect and/or isolate Neu5Gc expressing tumour-derived exosomes from a sample. Exosome capture was relatively consistent regardless of SubBΔS106/ΔT107 molecular net quantity demonstrating that sufficient Neu5Gc binding molecule was present to bind Neu5Gc expressed on the membrane surface of tumour-derived exosomes (Table 1).

TABLE 1
Mean fluorescent intensity of fluorescently labelled
MCF7 exosomes in wash and bound fractions and flow
through of the binding assay. The amount of MCF7 exosomes
was fixed (1 × 10⁷ exosomes/ml).

| | SubBΔS106/ΔT107 molecular net (fixed | | | | | | |
|---|---|---|---|---|---|---|---|
| | amount of MCF7 exosomes) | | | | | | |
| Volume of | 0 | 15 | μL | 30 | μL | 45 | μL |

| SubBΔS106/ΔT107 | | | | |
|---|---|---|---|---|
| molecular | | | | |
| TOTAL MFI | 28750 | 21996 | 19613 | 19641 |
| FT MFI | 25365 | 11217 | 9884 | 9336 |
| WABF MFI | 3385 | 10779 | 9729 | 10305 |

Table 2 shows the results of contacting a fixed amount of SubBΔS106/ΔT107 molecular net with varying amounts of MCF7 exosomes in a sample. When 50 μL of MCF7 exosomes were contacted with a fixed amount of SubBAS106/AT107 molecular net, approximately 52% of MCF7 exosomes were detected in the WABF compared to total MFI fraction of the binding assay (Table 2). The proportion of MCF7 exosomes detected in the WABF compared to total MFI fraction of the binding assay was approximately 44% and 36% when 100 μL and 200 μL of MCF7 exosomes were respectively contacted with a fixed amount of SubBΔS106/ΔT107 molecular net (Table 2). As expected, a low MFI (background signal) was detected in the control sample where MCF7 exosomes were not present (Table 2).

TABLE 2
Mean fluorescent intensity of fluorescently labelled
MCF7 exosomes in wash and bound fractions and flow
through of the binding assay. The amount of SubBΔS106/ΔT107
molecular net was fixed (30 μL).

| | MCF7 Exosomes (fixed amount of SubBΔS106/ΔT107 | | | | | | |
|---|---|---|---|---|---|---|---|
| | molecular net) | | | | | | |
| Volume of MCF7 | 0 | 50 | μL | 100 | μL | 200 | μL |

| | | | | |
|---|---|---|---|---|
| Exosomes | | | | |
| TOTAL MFI | 102 | 19308 | 36028 | 66369 |
| FT MFI | 18 | 9251 | 20293 | 42700 |
| WABF MFI | 84 | 10057 | 15735 | 23669 |

Tables 1 and 2 unexpectedly show that Neu5Gc is expressed on the membrane surface of tumor-derived exosomes and that this surface expression can be utilized to isolate and/or detect tumour-derived exosomes using a Neu5Gc binding molecule such as SubBΔS106/ΔT107.

The results of Tables 1 and 2 demonstrate that approximately 50% MCF7 derived exosomes were isolated using the SubBΔS106/ΔT107 molecular net. To confirm that the SubB2M molecular nets were capturing tumour-derived exosomes, the level of general biomarker for cancer miR-21 was analysed in isolated exosomes using qRT-PCR. The results are presented in Table 3.

TABLE 3
miR-21 analysis by qRT-PCR of the bound
fractions from the binding assays.

| | MCF7 Exosomes | | | | | | |
|---|---|---|---|---|---|---|---|
| | (fixed amount of SubBΔS106/ΔT107 molecular net) | | | | | | |
| Volume of MCF7 | 0 | 50 | μL | 100 | μL | 200 | μL |

| | | | | |
|---|---|---|---|---|
| Exosomes | | | | |
| Mean Ct Values | N/A | 35.353 | 35.426 | 34.477 |

As expected, miR-21 recovery was detected in all replicates comprising tumour-derived exosomes. No miR-21 was detected in control samples. These data confirm that Neu5Gc binding molecule can be used to isolate and/or detect tumour-derived exosomes in a form suitable for further analysis such as nucleic acid expression analysis.

### Example 4: Additional Neu5Gc Based Purification of Tumour-Derived Exosomes

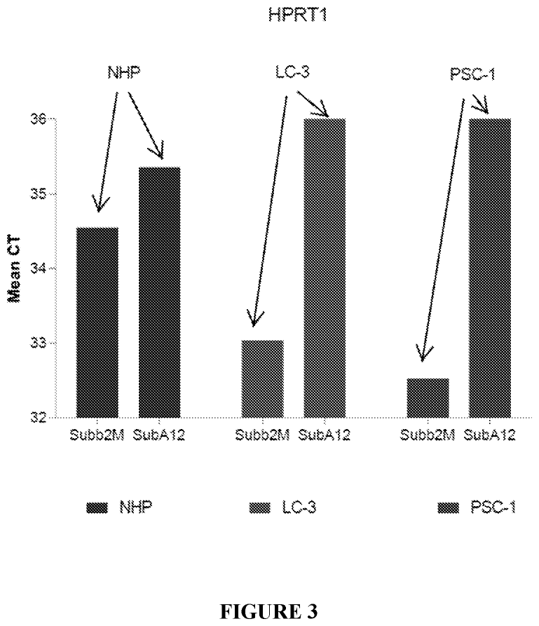

Example 4 further supports the tumour derived exosome capturing capability of Neu5Gc binding protein, SubB2M, evidenced in Examples 1 and 3. In this example, human plasma samples were obtained from patients with lung cancer, prostate cancer as well as healthy controls. 400-500 μL of respective plasma sample was contacted with 30 μL of either SubB2M (Neu5Gc binding protein) or SubA12 (binding protein which binds non-sialic acid component, but does not substantially bind Neu5Gc) presented on magnetic beads. HPRT1 mRNA levels were then assessed using qPCR to provide a measure of exosomes captured by SubB2M. Consistent with the results in Example 3, SubB2M captured significantly more HPRT1 mRNA from cancer plasma than normal plasma and this finding was consistent across both cancers tested (FIG. 3). Taken together with the results in Examples 1 and 3, these data further support a general concept for capturing extracellular vesicles from tumours based on their expression of Neu5Gc.

### Example 5: Cancer Exosome Detection

A bead coated with a layered, multipolymeric molecular net structure comprised of crosslinkers and antibodies directed against different surface epitopes of exosomes is contacted with a sample containing exosomes. Captured exosomes are then screened for the cancer biomarker, Neu5Gc, using either an anti-Neu5Gc antibody or SubBΔS106/ΔT107 mutant and an appropriate reporter for detecting binding to Neu5Gc.

### Example 6: Cancer Detection

A bead coated with a layered, multipolymeric molecular net structure comprised of crosslinkers and anti-Neu5Gc binding molecule is contacted with a sample containing exosomes that has been isolated from a patient suspected of having cancer. Binding of the Neu5Gc binding molecule to extracellular vesicles indicates the presence of tumour-derived extracellular vesicles in the sample.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

The present application claims priority from AU2021901359 filed 6 May 2021 the disclosures of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A method of isolating tumour-derived extracellular vesicles from a sample, the method comprising:
contacting a sample comprising tumour-derived extracellular vesicles with a binding molecule that binds to N-glycolylneuraminic acid (Neu5Gc) under conditions that enable binding of the binding molecule to the tumour-derived extracellular vesicles in the sample; and
isolating the binding molecule from the sample.

2. (canceled)

3. The method according to claim 1, wherein the sample is contacted with a layered, multipolymeric molecular net which comprises multiple layers of the binding molecule.

4. A method of detecting tumour-derived extracellular vesicles comprising:
contacting a sample comprising extracellular vesicles with a binding molecule that binds to N-glycolylneuraminic acid (Neu5Gc); and,
detecting binding of the Neu5Gc binding molecule to extracellular vesicles in the sample, wherein binding of the Neu5Gc binding molecule to extracellular vesicles indicates the presence of tumour-derived extracellular vesicles in the sample.

5. A composition comprising extracellular vesicles enriched for extracellular vesicles expressing N-glycolylneuraminic acid (Neu5Gc), wherein the extracellular vesicles are tumour-derived extracellular vesicles isolated by the method of any claim 1.

6. A population of extracellular vesicles enriched for extracellular vesicles expressing Neu5Gc, wherein the extracellular vesicles are tumour-derived extracellular vesicles isolated by the method of claim 1.

7. The composition of claim 5, wherein:
(i) at least 20% of the extracellular vesicles in the composition or express N-glycolylneuraminic acid (Neu5Gc);
(ii) at least 50% of the extracellular vesicles in the composition express N-glycolylneuraminic acid (Neu5Gc); or
(iii) at least 30%, at least 40%, at least 60%, or at least 70% of the extracellular vesicles in the composition are extracellular vesicles expressing N-glycolylneuraminic acid (Neu5Gc).

8. (canceled)

9. (canceled)

10. The method according to claim 1, wherein the sample is obtained from a subject suspected of having cancer or a subject that has cancer.

11. The method according to claim 1, wherein the sample:
(i) is selected from a group consisting of blood, plasma, serum, urine, saliva, faeces, tears, brocho-alveolar lavage fluid (BALF), cerebrospinal fluid (CSF) and seminal fluid;
(ii) is a purified or partially purified population of extracellular vesicles, and optionally wherein the purified or partially purified population of extracellular vesicles express one or more protein(s) selected from a group consisting of CD63, b2microglobulin, CD11, CD81, CD13, and EGFR;
(iii) is substantially free of cells; and/or
(iv) is the supernatant of a tumour cell culture.

12. (canceled)

13. (canceled)

14. (canceled)

15. (canceled)

16. The method according to claim 1, wherein the binding molecule is an isolated protein comprising an amino acid sequence as set forth in SEQ ID NO: 2 or SEQ ID NO: 4 or fragment or variant of SEQ ID NO: 2 or SEQ ID NO: 4 which comprises a modification to at least one of the amino acid residues in SEQ ID NO: 2 or SEQ ID NO: 4, and, wherein the fragment or variant thereof is capable of binding α2-3-linked N-glycolylneuraminic acid and α2-6-linked N-glycolylneuraminic acid.

17. The method of claim 16, wherein the modification comprises a non-conservative substitution or deletion of at least one of the underlined residues of TTSTE from SEQ ID NO: 1.

18. The method according to claim 1, wherein the binding molecule comprises:
(i) a single chain Fv fragment (scFv);
(ii) a dimeric scFv (di-scFv);
(iii) a diabody;
(iv) a triabody;
(v) a tetrabody;
(vi) a nanobody;
(vii) a Fab;
(viii) a F(ab′)₂;
(ix) a Fv;
(x) an aptamer;
(xi) one of (i) to (ix) linked to a constant region of an antibody, Fc, or a heavy chain constant domain (C_{H}) 2 and/or C_{H}3;
(xii) one of (i) to (ix) linked to albumin or a functional fragment or variants thereof or to a protein that binds to albumin; or
(xiii) an antibody.

19. The method of claim 1, wherein the binding molecule is an antibody.

20. The method according to claim 1, wherein the method further comprises dissociating bound tumour-derived extracellular vesicles from the binding molecule and collecting the dissociated tumour-derived extracellular vesicles, optionally wherein the method further comprises analysing the tumour-derived extracellular vesicles.

21. (canceled)

22. The method of claim 10, wherein the cancer is selected from a group consisting of breast cancer, melanoma, malignant epithelial tumour, oesophageal carcinoma, gastric cancer, colorectal cancer, epidermoid carcinoma of rectum, pancreatic cancer, hepatocellular carcinoma, lymph node metastases, kidney cancer, urinary bladder cancer, ovarian cancer, uterine cancer, testicular cancer, prostate cancer, neuroblastoma, non-small cell lung cancer, lymphoma, neuroectodermal tumour (astrocytoma and glioblastoma), nephroblastoma (Wilms tumours), sarcoma, Ewing sarcomas and thyroid carcinoma.

23. (canceled)

24. The method according to claim 1, wherein the extracellular vesicles are selected from a group consisting of small extracellular vesicles, exosomes, exomeres and microvesicles.

25. The method according to claim 1, wherein the extracellular vesicles are exosomes, and optionally wherein the extracellular vesicles are CD63+ exosomes.

26. (canceled)

27. A method of detecting cancer comprising:
contacting a sample comprising extracellular vesicles with a binding molecule that binds to N-glycolylneuraminic acid (Neu5Gc); and
detecting binding of the Neu5Gc binding molecule to extracellular vesicles in the sample, wherein binding of the Neu5Gc binding molecule to extracellular vesicles indicates the presence of tumour-derived extracellular vesicles in the sample, thereby detecting cancer.

28. The method of claim 27, wherein the binding molecule comprises an amino acid sequence as set forth in SEQ ID NO: 2 or SEQ ID NO: 4.

29. The population according to claim 6, wherein the extracellular vesicles are exosomes, and optionally wherein the extracellular vesicles are CD63+ exosomes.


SEQUENCE LISTING (4 sequences)

SEQ ID NO: 1 - protein, 121 aa, Escherichia coli
  native B subunit of AB5
AMAEWTGDARDGMFSGVVITQFHTGQIDNKPYFCIEGKQSAGSSISACSMKNSSVWGASFSTLYNQALYFYTTGQPVRIYYKPGVWTYPPFVKALTSNALVGLSTCTTSTECFGPDRKKNS

SEQ ID NO: 2 - protein, 116 aa, Artificial Sequence
  SubBdeltaS106/deltaT107 mutant
EWTGDARDGMFSGVVITQFHTGQIDNKPYFCIEGKQSAGSSISACSMKNSSVWGASFSTLYNQALYFYTTGQPVRIYYKPGVWTYPPFVKALTSNALVGLSTCTTECFGPDRKKNS

SEQ ID NO: 3 - protein, 5 aa, Artificial Sequence
  glycan binding motif of the native B subunit of AB5
TTSTE

SEQ ID NO: 4 - protein, 116 aa, Artificial Sequence
  SubB2M
EWTGDARDGMFSGVVITQFHTGQIDNKPYFCIEGKQSAGSSISACSMKNSSVWGASFSTLYNQALYFYTTGQPVRIYYEPGVWTYPPFVKALTSNALVGLSTCTTECFGPDRKKNS